(19) United States
(12) Patent Application Publication
(10) Pub. No.: US 2011/0206737 A1
(43) Pub. Date: Aug. 25, 2011

(54) PHOTOSENSITIZER-CONTAINING COMPOSITION

(75) Inventor: Sung-Wei CHEN, Singapore (SG)

(73) Assignee: EMPIRE TECHNOLOGY DEVELOPMENT LLC, Wilmington, DE (US)

(21) Appl. No.: 12/711,271

(22) Filed: Feb. 24, 2010

(51) Int. Cl. A61K 9/00; C01B 13/02; B32B 7/02; A61P 35/04

(52) U.S. Cl. 424/400; 252/186.1; 428/212; 204/157.5

(57) ABSTRACT

Techniques related to a photosensitizer-containing composition, the preparation method and the applications thereof, are generally described. One example photosensitizer-containing composition may include a carrier; and a photosensitizer attached to the surface of the carrier, wherein the carrier includes a first material having a first refractive index and a second material having a second refractive index, and the first refractive index is greater than the second refractive index.

### BACKGROUND

Photodynamic therapy (PDT) is a treatment for cancer. PDT involves three key components: a PDT composition, light, and molecular oxygen in tissues. The PDT composition is configured to be administered to tissues affected by cancer. The PDT composition is further configured to absorb light and to be excited from a ground singlet state to an excited singlet state. In the excited singlet state, the PDT composition may be relaxed to an excited triplet state, and the resulting energy is absorbed by the molecular oxygen. The molecular oxygen is then excited to produce singlet oxygen molecules. Singlet oxygen is a very aggressive chemical species and rapidly reacts with any nearby tissues to treat cancer. It may also be used to treat infections by micro-organisms and other diseases. However, typical PDT compositions have adoption issues because of their optical properties.

### SUMMARY

One embodiment of the disclosure may generally relate to a photosensitizer-containing composition. The photosensitizer-containing composition may include a carrier, and a photosensitizer attached to the surface of the carrier, where the carrier includes a first material having a first refractive index and a second material having a second refractive index, and the first refractive index is greater than the second refractive index.

Another embodiment of the disclosure may generally relate to a method for making a photosensitizer-containing composition. The method may include attaching, a photosensitizer onto a surface of a carrier, where the carrier includes a first material having a first refractive index and a second material having a second refractive index, and the first refractive index is greater than the second refractive index.

Yet another embodiment of the disclosure may generally relate to a method for generating singlet oxygen with a photosensitizer-containing composition. The method may include directing light to the photosensitizer-containing composition in the presence of molecular oxygen, wherein the photosensitizer-containing composition includes a photosensitizer and a carrier including a first material having a first refractive index and a second material having a second refractive index, and the first refractive index is greater than the second refractive index, and wherein an interaction of the light with the carrier enhances the energy transfer of light by the photosensitizer to produce an excited photosensitizer, and wherein the excited photosensitizer transfers energy to the molecular oxygen to produce singlet oxygen.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a structure of an illustrative embodiment of a photodynamic therapy composition;

FIG. 2 is a flow chart of an illustrative embodiment of a method for making a photodynamic therapy composition; and

FIG. 3 is a flow chart of an illustrative embodiment of a method for using a photodynamic therapy composition.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is drawn, inter alia, to a photosensitizer-containing composition, methods of making the composition, and applications of use related to the photosensitizer-containing composition.

The photosensitizer-containing composition described herein contains a carrier and a photosensitizer attached, e.g., absorbed, ionically associated or covalently bonded, to the surface of the carrier. The carrier may include at least two materials having two different refractive indices. In some embodiments, the carrier contains a first material having a first refractive index and a second material having a second refractive index, and the first refractive index is greater than the second refractive index.

Methods of making the composition are also described herein. The methods include attaching a photosensitizer onto a surface of a carrier, for example, by absorbing, ionically associating or covalent bonding. The carrier may be formed by layering a first material having a first refractive index with a second material having a second refractive index. The first refractive index is greater than the second refractive index.

Methods of using the composition are also described herein. One method includes directing a light beam to the photosensitizer-containing composition including a photosensitizer and a carrier; and exciting the photosensitizer with the light. The carrier includes a first material having a first refractive index and a second material having a second refractive index, and the first refractive index is greater than the second refractive index. The energy transferred from the light is increased because of the structure of the carrier. Energy received by the photosensitizer is transferred to molecular oxygen to produce singlet oxygen. In some embodiments, energy received by the photosensitizer compound may be transferred through intermediates (Type I reaction) or directly (Type II reaction) to molecular oxygen to produce singlet oxygen. In some embodiments, the singlet oxygen generated in this process may be used as a therapeutic agent. For example, the compositions described herein may be used in a method for treatment of a malignant tumor by photodynamic therapy.

In some embodiments, the carrier may be a photonic bandgap material. In this disclosure, a “photonic bandgap material” may generally refer to a periodic optical structure that is designed to affect the motion of photons similar to the way in which periodicity of a semiconductor crystal affects the motion of electrons. The term “photonic bandgap material” is intended to refer to materials referred to the term “photonic crystal” in the art.

The photonic bandgap material may be a one dimensional photonic bandgap material. A one dimensional photonic bandgap material may be a photonic bandgap material in a form of periodic multi-layered dielectric stacks. The photonic bandgap material may include a first material having a first refractive index and a second material having a second refractive index. The photonic bandgap material may be a layered structure formed by alternating layers of the first material and the second material. The first refractive index may be greater than the second refractive index. The thickness of the second material in the photonic bandgap material may be greater than the thickness of the first material in the photonic bandgap material.

Some example combinations of the first material and the second material of the compositions described herein, for example, a photonic bandgap material, may include, without limitation, titanium dioxide and silicon dioxide, zirconium dioxide and silicon dioxide, hafnium dioxide and silicon dioxide, tellurium and polystyrene, tin sulfide and silicon dioxide, or poly vinylcarbazole and polyvinyl alcohol, respectively.

The photosensitizer in the compositions describe herein may include any technically feasible photosensitizer capable of transferring absorbed energy to molecular oxygen to generate singlet oxygen. Some example photosensitizers may include, without limitation, porfimer sodium, prodrug-aminolevulinic acid photosensitizer protoporphyrin IX, methyl aminolevulate photosensitizer protoporphyrin IX, hexyl aminolevulate photosensitizer protoporphyrin IX, benzoporphyrin derivative monoacid A, tetra_m-hydroxyphenyl_chlorin, motexafin lutetium, Pd-bacteriopheophorbide, taloporfin sodium or silicon pthalocyanine 4.

In embodiments in which the composition contains a photonic bandgap material with layers of the first and second material, light may be absorbed at a bottom layer of the photonic bandgap material. In response to the light, the photonic bandgap material may: 1) create total internal reflection for the light wavelength, and/or 2) generate an enhanced evanescent field for the light wavelength at the interface of the top layer and the photosensitizer. The photosensitizers attached, e.g., absorbed, ionically associated or covalently bonded, onto the top layer of the photonic bandgap material may be excited by these effects and/or the incident light.

A method for making a photosensitizer-containing composition is also provided. In some embodiments, a first material having a first refractive index and a second material having a second refractive index may be used to form a carrier, for example, a photonic bandaap material. The carrier, for example, a photonic bandgap material, may be a layered structure having alternating layers of the first material and the second material. The carrier, for example, a photonic bandgap material, may be formed by any technically feasible approach. Example approaches may include, without limitation, sputtering, electrodeposition, or other thin-film methods. The thickness of the first material and the thickness of the second material may be controlled. In some embodiments, the thickness of the material having a greater refractive index is thinner than the thickness of the material having a smaller refractive index. The formed carrier, for example, photonic bandgap material, may be milled or ground into small particles. In some embodiments, the diameter of the particles may be about 500 microns or less.

A photosensitizer may be functionalized and covalently attached to the surface of the ground carrier, e.g., photonic bandgap material. The photosensitizer may be functionalized so that the photosensitizer and the surface of the carrier may be conjugated together. The functional groups to be added to the photosensitizer are based on the chemical structures of the photosensitizer and the carrier. A person skilled in the art may select and add appropriate functional groups to the photosensitizer. Some possible functional groups may include, without limitation, hydroxyl groups, sulfhydryl groups, amino groups, carboxyl groups, or aldehyde groups. In some other embodiments, the carrier surface may be functionalized as well for conjugation to the photosensitizer.

Applications of a manufactured photosensitizer-containing composition as described herein are also provided. The photosensitizer-containing composition includes a carrier and a photosensitizer. In some embodiments, the carrier may be a photonic bandgap material as described herein. The photosensitizer-containing composition is introduced into an organism, for example, a mammal, such as a human, and exposed to a light beam of a certain wavelength. In embodiments in which the carrier is a layered photonic bandgap material as described herein, a light beam may be incident on a lower layer of the photonic bandgap material, and the photonic bandgap material may cause an enhancement of the energy transfer from the light excitation by the photosensitizer at the given wavelength.

The photosensitizer may be excited by a light having a specific range of wavelengths to generate singlet oxygen. In some embodiments, some example photosensitizer may be excited by a light having a wavelength from about 630 nm to about 765 nm. In some other embodiments, some other example photosensitizer may be excited by a light having a wavelength about 405 nm. The photosensitizer-containing composition may be engineered to provide enhancement of energy transfer to the photosensitizer at a wavelength that falls within the specific range. Some factors may affect the enhancement level. Example factors may include, without limitation, the two materials of the carrier, for example, photonic bandgap material, having two different refractive indices, or the relative thicknesses of the two materials of the carrier. After the photosensitizer is excited, singlet oxygen may be generated as set forth above. The singlet oxygen may react with nearby cells to destroy malignant tissue.

The photosensitizer-containing composition may be used in photodynamic therapy. In some embodiments, a method for photodynamic therapy may include administering a photosensitizer-containing composition to an individual in need thereof. The administering may be based on the health conditions of the individual. The composition may comprise a photosensitizer and a carrier. The carrier may include a first material having a first refractive index and a second material having a second refractive index, and the first refractive index is greater than the second refractive index.

The composition may be administered to a tissue comprising a tumor, and illuminated with light. Enhanced energy transfer of the light energy to the photosensitizer may be induced by the carrier, producing an excited photosensitizer. The excited photosensitizer transfers energy to the molecular oxygen in the tissue to produce singlet oxygen.

The composition may also be used as a therapeutic substance. More specifically, the composition may be used in treating a malignant tumor or an infection by a micro-organism. The composition may be used for manufacture of a medicament for treating a malignant tumor as well. In some embodiments, the composition may be administered into tissues of the malignant tumor or the micro-organism. The molecular oxygen contained in the tissues or the micro-organisms may generate singlet oxygen with an energy received from the photosensitizer.

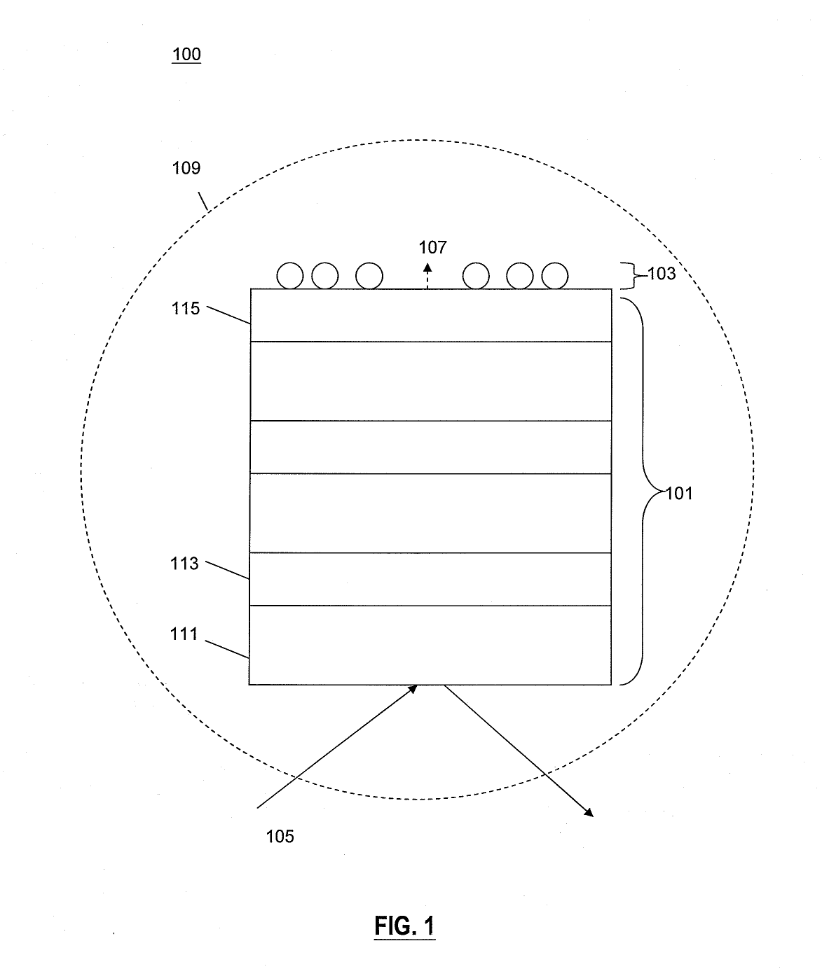

FIG. 1 shows a structure of an illustrative embodiment of a photodynamic therapy composition 100. As depicted, the photodynamic therapy composition 100 includes a photonic bandgap material 101 and a photosensitizer 103, and the photodynamic therapy composition 100 is in an environment 109. The photonic bandgap material 101 may be formed by alternating layers of a material 111 and a material 113, wherein the material 111 has a first refractive index and the material 113 has a second refractive index. In some implementations, the second refractive index is greater than the first refractive index and the thickness of the material 111 is thicker than the thickness of the material 113.

For example, the material 111 may be polystyrene, the material 113 may be tellurium, and the environment 109 may be water. The refractive index of polystyrene is about 1.6, the refractive index of tellurium is about 4.6, and the refractive index of water is about 1.3. The thickness ratio between a polystyrene layer and a tellurium layer is about 2. For example, the thickness of a polystyrene layer may about 93.4 nm and the thickness of a tellurium layer may be about 46.7 nm. A light beam 105 may be directed to a bottom surface of the photonic bandgap material 101 (e.g., as depicted in FIG. 1, a bottom surface of the lowest layer of the material 111), resulting in enhanced energy transfer 107 at a top surface of a layer 115 of the photonic bandgap material 103. Depending on the alternating structure of the material 111 and material 113, the lowest layer of the photonic bandgap material 101 may either be a layer of the material 111 or the material 113, and layer 115 may either be a layer of the material 111 or the material 113.

In some implementations, the photonic bandgap material 101 may be carried onto a glass substrate. The glass substrate may be arranged below the bottom surface of the photonic bandgap material 101 (e.g., as depicted in FIG. 1, the bottom surface of the lowest layer of the material 111).

The light source of the light beam 105 may be selected to generate a light beam 107 having an appropriate wavelength to excite the photosensitizer 103. The material 111, the material 113, and their respective thicknesses may be adjusted as well to achieve the same goal.

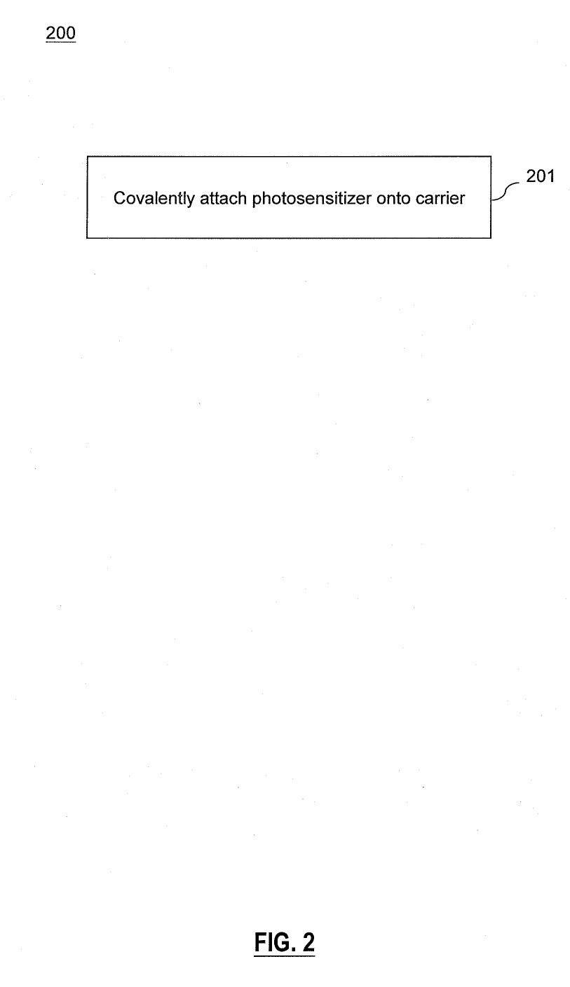

FIG. 2 is a flow chart of an illustrative embodiment of a method 200 for making a photodynamic therapy composition. The method 200 may begin at block 201 (covalently attach photosensitizer onto carrier), where a photosensitizer is covalently attached onto a surface of a carrier. In some implementations, the carrier may be a one dimensional photonic bandgap material as set forth above. Based on the chemical structures of the photosensitizer and the one dimensional photonic bandgap material, an appropriate conjugation mechanism may be used to covalently attach the photosensitizer onto the surface of the photonic bandgap material.

For example, the photosensitizer may be a porphyrin derived compound. One material in the photonic bandgap material may be poly vinyl alcohol. Both the porphyrin derived compound and poly vinyl alcohol have unsaturated bonds for conjugation. A person skilled in the art may conjugate the two materials based on known approaches. One example approach may include functionalizing porphyrin derived compound or poly vinyl alcohol with appropriate functional groups to facilitate the conjugation.

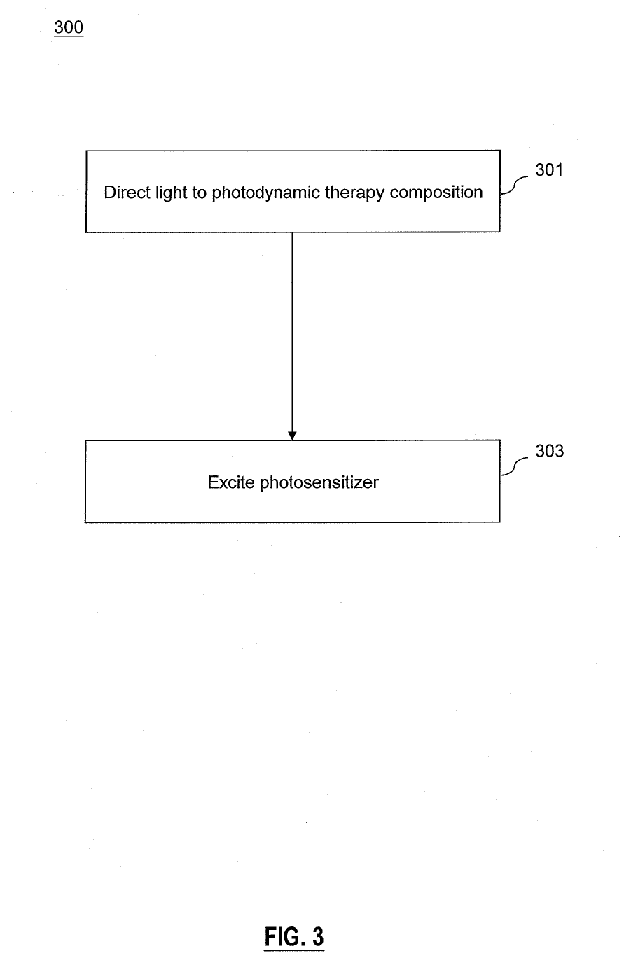

FIG. 3 is a flow chart of an illustrative embodiment of a method 300 for using a photodynamic therapy composition. The method 300 may begin at block 301 (direct light to photodynamic therapy composition), where light is directed to a photodynamic therapy composition. The photodynamic therapy composition may include a photosensitizer and a photonic bandgap material. The photonic bandgap material may interact with the light and, as a result, enhance the energy transfer to the photosensitizer. The photosensitizer covalently attached onto the surface of the photonic bandgap material is excited by the enhanced energy transfer block 303 (excite photosensitizer).

In some other implementations, the method 300 may further include administering the photodynamic therapy composition to a cancer affected or infected tissue. A light beam is configured to be directed to the photodynamic therapy composition, and the resulting enhanced energy transfer by the photonic bandgap material excites the photosensitizer to facilitate the generation of singlet oxygen. The singlet oxygen may react with the cancer affected tissue to treat cancer, for example, to destroy malignant cells in a tumor; or infected tissue to treat the infection, for example, to destroy the infectious micro-organism.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims; or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

I/We claim:

1. A photosensitizer-containing composition, comprising:
a carrier; and
a photosensitizer attached to the surface of the carrier, wherein the carrier includes a first material having a first refractive index and a second material having a second refractive index, and the first refractive index is greater than the second refractive index.

2. The photosensitizer-containing composition of claim 1, wherein the carrier has a layered structure with alternating layers of the first material and the second material.

3. The photosensitizer-containing composition of claim 1, wherein the first material has a first thickness in the carrier and the second material has a second thickness in the carrier, and the second thickness is greater than the first thickness.

4. The photosensitizer-containing composition of claim 1, wherein the carrier is a one dimensional photonic bandgap material.

5. The photosensitizer-containing composition of claim 4, wherein the carrier further includes a glass substrate in parallel with the layers of the first material and the layers of the second material, and wherein the carrier is deposited onto the glass substrate.

6. A method for making a photosensitizer-containing composition, comprising:
attaching a photosensitizer onto a surface of a carrier to form the photosensitizer-containing composition, wherein the carrier includes a first material having a first refractive index and a second material having a second refractive index, and the first refractive index is greater than the second refractive index.

7. The method of claim 6, further comprising functionalizing the surface of the carrier prior to attaching the photosensitizer to the surface of the carrier.

8. The method of claim 6, further comprising functionalizing the photosensitizer prior to attaching the photosensitizer to the surface of the carrier.

9. The method of claim 6, further comprising processing the carrier so that the carrier has a diameter of about 500 microns or less prior to attaching the photosensitizer to the surface of the carrier.

10. A method for generating singlet oxygen with a photosensitizer-containing composition, comprising:
directing light to the photosensitizer-containing composition in the presence of molecular oxygen,
wherein the photosensitizer-containing composition comprises a photosensitizer and a carrier, wherein the carrier includes a first material having a first refractive index and a second material having a second refractive index, and the first refractive index is greater than the second refractive index,
wherein an interaction of the light with the carrier enhances the energy transfer of light by the photosensitizer to produce an excited photosensitizer,
and wherein the excited photosensitizer transfers energy to the molecular oxygen to produce singlet oxygen.

11. The method of claim 10, wherein the light is absorbed at a first layer of the carrier and the enhancement of energy transfer of light energy occurs at an interface of the photosensitizer and a second layer of the carrier, wherein the first layer and the second layer are at different sides of the carrier and in parallel with each other.

12. The method of claim 11, wherein an evanescent field is generated at the second layer.

13. The method of claim 12, wherein the energy transfer of light to the photosensitizer is enhanced by the evanescent field in comparison with a photosensitizer without the presence of the carrier.

14. The method of claim 10, wherein the carrier creates total internal reflection of the light.

15. The method of claim 14, wherein the energy transfer of light to the photosensitizer is enhanced by the total internal reflection in comparison with a photosensitizer without the presence of the carrier.

16. The method of claim 10, further comprising administering the photosensitizer-containing composition to a tissue affected by a disease or an infection prior to directing light to the photosensitizer-containing composition.

17. The method of claim 16, wherein the singlet oxygen is generated from the molecular oxygen in the tissue after the molecular oxygen in the tissues receives energy transferred from the excited photosensitizer.

18. The method of claim 17, where the disease or the infection is cancer.

19. The method of claim 17, where the disease or the infection is caused by a micro-organism.

20. The method of claim 19, wherein the micro-organism comprises molecular oxygen, and the singlet oxygen is generated from the molecular oxygen after the molecular oxygen receives energy transferred from the excited photosensitizer.

21. The method of claim 10, further comprising administering the photosensitizer-containing composition to a micro-organism prior to directing light to the photosensitizer-containing composition.
